(19) United States
(12) Patent Application Publication
(10) Pub. No.: US 2020/0249229 A1
(43) Pub. Date: Aug. 6, 2020

(54) BODILY PROCESSING OF ACTIVITY SENSORS

(71) Applicant: GLYMPSE BIO, INC., Cambridge, MA (US)

(72) Inventors: Sangeeta Bhatia, Lexington, MA (US); Gabriel Kwong, Atlanta, GA (US); Eric Huang, Lexington, MA (US); Sirshendu Roopom Banerjee, Lexington, MA (US); Andrew David Warren, Cambridge, MA (US); Sophie Cazanave, Cambridge, MA (US)

(21) Appl. No.: 16/776,991

(22) Filed: Jan. 30, 2020

Related U.S. Application Data
(60) Provisional application No. 62/800,107, filed on Feb. 1, 2019.

(51) Int. Cl. G01N 33/569; G01N 33/543

(52) U.S. Cl. CPC G01N 33/56966; G01N 33/54393; G01N 33/54313

(57) ABSTRACT

The invention provides methods and compositions that use a process in the body to deliver an active form of the activity sensor to a target site within the body. The activity sensors, which release detectable reporters when acted on by certain enzymes within the body, are provided as pro-analytes. Processes within the body deliver the activity sensors in active form to a target site of interest. For example, enzymes or a chemical environment within the body may cleave blocking groups from the activity sensors, or the body's tissues and organs may collect the activity sensors at the target site based on size or composition of the activity sensors.

### TECHNICAL FIELD

The disclosure generally relates to activity sensors that can be delivered into a body and that report activity within the body via biochemical/chemical reactions that result in the release of detectable reporters.

### BACKGROUND

Current approaches to detecting or diagnosing diseases typically require taking a sample from the body and processing the sample for further analysis. For example, some approaches to cancer detection and monitoring involve sequencing tumor DNA detect cancer-specific mutations. Obtaining the sample is problematic for various reasons. Needle biopsies require inserting a needle deeply into a patient to obtain a sample of disease-affected cells. Furthermore, if the needle biopsy misses the tumor and only retrieves healthy tissue, the procedure will produce a false-positive result.

Not only can obtaining the sample be painful to the patient and a point of analytical failure, it is only the beginning of the laboratory work required to produce clinically-actionable information. Analysis of cancer-specific mutations requires extensive sample preparation to extract DNA, isolate relevant DNA fragments, and amplify those fragments with adaptors and reagents required for DNA sequencing. If the DNA sample prep and sequencing proves successful, then it is hoped that the results will provide a report of tumor-specific mutations that offers some insight into the nature of the underlying disease. In all of these techniques, the sample prep is a potential point of failure. Thus, there is a desire for minimal sample prep in order to increase the sensitivity and specificity of diagnostic tests.

### SUMMARY

The invention provides methods that use a patient's own body to prepare a sample for analysis. In methods of the invention, a pro-analyte composition is administered. The pro-analyte composition comprises detectable reporters that are concentrated under specific conditions within the body. The invention takes advantage of the substantially unique condition of an organ system in the presence of disease in order to produce analytes that are concentrated in amounts detectable over background only in the presence of the disease state.

In one preferred embodiment, pro-analytes are converted into an analyte that is enriched for detection. For example, a hydrophobic moiety that passes freely through cell membranes is converted into a hydrophilic moiety that allows it to be concentrated and enriched in a cell. The conversion can be enzymatic or by means of another chemical reaction in the cell. In another preferred embodiment, detectable reporters are cleaved from the pro-analytes under conditions present in a diseased tissue but not in healthy tissue. One way in which the invention takes advantage of the body's ability to act as a sample preparation vehicle is by introducing pro-analytes that are cleaved by enzymes uniquely present in organ systems having a particular condition. Many disease states are characterized by the presence of unique enzymes or an increase in the production of certain enzymes. Released detectable reporters are obtained from a body sample, such as urine, blood, sputum, a tissue sample, saliva, or even an exhalant (i.e., breath), and measured to identify a condition. The detectable reporters can be quantitatively measured to indicate more specifically the health of the patient. In addition to reporting on a disease state, pro-analytes can be tuned to report on overall health, including indicia of healthy levels of enzymes or other biomarkers present in an organ system. However they are used, the invention provides methods and compositions in which, the body performs the sample preparation steps to yield a measurable result.

Pro-analyte compositions of the invention can be in the form of activity sensors that include substrates for a set of enzymes know to be expressed under specific conditions. While the condition can be overall health status (i.e., indicative of either good or bad health), the primary use of the invention is via interaction of activity sensors with disease-specific enzymes in a particular organ or bodily compartment that result in the release of detectable reporters via cleavage of linkers between the reports and the activity sensors. The pro-analyte is designed and composed such that it only releases the detectable reporter upon encountering disease-specific conditions in the affected organ or body compartment. For example, an activity sensor can target cancer in a specific organ system by designing the pro-analyte to include a plurality of peptides that act as substrates for proteases known to be active when the specific type of cancer is present. Only in the presence of those enzymes is the pro-analyte processed to release quantities of detectable markers (e.g., various peptide fragments). Thus, detecting those peptide fragments in a sample, such as a urine sample, from the patient indicate the presence of the cancer in the organ.

By taking advantage of the “body as sample prep”, methods of the disclosure use activity sensors to probe the physiological state of tissue or a compartment non-invasively within the body. Numerous physiological states of interest, such as various specific diseases, are characterized by the activity of enzymes associated with a particular health state, such as a disease, syndrome, or condition. When condition-specific enzymes cleave the activity sensors to release the detectable reporters, detection of those reporters in samples from the body indicates the physiological state of the tissue. Of note, many diseases involve mechanisms of action by which enzyme activity is dysregulated prior to the appearance of other symptoms. For example, neoplastic cells will release extracellular tissue remodeling enzymes to cleave the extracellular matrix before the cells proliferate into a detectable tumor and before those tumor cells release a level of tumor DNA fragments that can be detected by liquid biopsy. Accordingly, the detection of extracellular protease activity at a specific site of interest can detect incipient cancer much earlier than the cancer could be detected by other methods. The activity sensors serve as accurate and precise sensors of activities that signify a particular physiological state of tissue or a body fluid.

In some aspects, the invention provides analysis methods that include the steps of: administering a pro-analyte to a patient, wherein a health condition of the patient is associated with a release of detectable reporters from the pro-analyte; measuring the detectable reporters in a sample from the patient; and correlating the measured reporters to a state of the health condition in the patient. The pro-analyte is preferably processed through multiple organ systems, resulting in release of the detectable reporters, and the methods also include providing a report comprising health information concerning the patient. The detectable reporters may be released in the presence of a specific disease state, wherein said disease state is organ-specific. For example, the disease may be selected from an inflammatory disease, cancer, a neurological disease, and liver disease. Optionally, the sample is selected from an exhalant, urine, blood, saliva, stool, tissue, and sputum.

In some embodiments, the detectable reporters comprise fragments released upon cleavage from the pro-analyte in the presence of the disease state. The cleavage may be enzymatic cleavage.

The reporters may include peptides, nucleic acids, lipids, or carbohydrates. The measuring step preferably includes quantifying released reporters.

In certain embodiments, the administering step comprises administering a first composition comprising a first set of pro-analytes and administering a second composition comprising a second set of pro-analytes to the patient. Release of detectable reporters from the first set of pro-analytes may be indicative of activity at a first organ, tissue, or bodily compartment, and release of detectable reporters from the second set of pro-analytes may be indicative of activity at a second, organ, tissue, or bodily compartment. Methods of the invention preferably proceed via performing non-invasive analysis of the patient.

In some embodiments, the pro-analyte is a carrier comprising a linker connected to one or more of said detectable reporters. The carrier may be, for example, a multi-arm polyethylene glycol scaffold. Preferably said detectable reporters are released via a condition-specific chemical reaction or chemical environment in the patient. The condition-specific chemical reaction may be one that occurs in excess in the presence of disease but that is substantially absent in a healthy organ. Optionally, the chemical reaction is an enzymatic reaction catalyzed by an enzyme that is present in a disease state but substantially absent in a healthy condition. The chemical reaction or environment may be one that occurs primarily in a diseased cell.

The pro-analyte may be administered as: a plurality of a first particle, each first particle comprising a plurality of a first detectable reporter releasably attached to a carrier; a plurality of a second particle, each second particle comprising a plurality of a second detectable reporter releasably attached to a carrier; and a plurality of a third particle, each third particle comprising a plurality of a third detectable reporter releasably attached to a carrier. The reporters may be attached to the particles by a peptide comprising a respective cleavage site of an enzyme, in which at least one of the cleavage sites is cleaved by an enzyme that exhibits greater activity under a disease state than under a non-disease state.

In certain aspects, the disclosure provides methods of preparing a sample for analysis. Methods include introducing a pro-analyte into a patient and allowing the body to process the pro-analyte in a site-specific manner to release a detectable reporter. A sample is taken from the body and methods include measuring the detectable reporter in the sample to identify a condition in the patient. The detectable reporter may include a plurality of peptide fragments that are released upon cleavage of the pro-analyte by a respective plurality of enzymes that are present in a site within the body under the specified condition. The detectable reporter may be measured by quantifying the peptide fragments in the sample. Preferably, the body processes the pro-analyte by means of a plurality of enzymes that are unique to the condition being measured, such as a number of extracellular proteases that are released under a certain disease condition. The body processes the pro-analyte by enzymatic cleavage to release the detectable reporter and the detectable reporter may be a plurality of peptide fragments detectable by an assay of the sample. The detectable reporters may be amino acids, peptides, nucleic acids, sugars, carbohydrates, lipids, or any other molecule that is releasable under prescribed conditions and is detectable in a biological sample.

In some embodiments, the condition is a specific disease, syndrome, or condition. The body processes the pro-analyte at the site to release the detectable reporter in quantities measurably different than quantities at which the reporter is released, if at all, elsewhere in the body or in the absence of the disease. In certain embodiments, the pro-analyte is administered as a composition that includes a plurality of nanoparticle activity sensors that release the detectable reporter in the presence of a plurality of cognate enzymes. Preferably, the detectable reporters include a plurality of distinct peptide fragments. Each activity sensor may include a plurality of polypeptides that are cleaved by one of the enzymes to release the detectable reporter. The enzymes may include one or more of the enzymes that exhibit elevated activity in association with a disease state. The enzymes may also include at least one enzyme not specific to the diseases state, to give a “control” signal or establish a background level of enzymatic activity. In certain embodiments, each activity sensor includes a number (e.g., between about two and about twelve) copies of only one polypeptide that is cleaved by its respective cognate enzyme. Each polypeptide may be linked to a carrier, such as a suitable molecular scaffold or core. In some embodiments, the carrier is a multi-arm polyethylene glycol (PEG) scaffold to which each polypeptide is covalently linked. The PEG scaffold carrier may have a mass between about 20 and 50 kDa, and each of the distinct peptide fragments may have a characteristic mass-to-charge ratio.

Other embodiments are within the scope of the disclosure. For example, some embodiments use the body as sample prep to process a pro-analyte and release an active form of activity sensor at a target site within the body. In some embodiments, compositions and methods use a process in the body to deliver an active form of the activity sensor to a target site within the body. The activity sensors, which release detectable reporters when acted on by certain enzymes within the body, are provided as pro-analytes. Processes within the body deliver the activity sensors in active form to a target site of interest. For example, enzymes or a chemical environment within the body may cleave blocking groups from the activity sensors, or the body's tissues and organs may collect the activity sensors at the target site based on size or composition of the activity sensors. In another example, the activity sensors may include ligands for receptors that are characteristic of the target site, or antibody domains cognate to antigens of a target tumor, to promote accumulation or intracellular uptake of the activity sensor at the target site.

In some aspects, the disclosure provides a method for evaluating a physiological state of a target site within tissue or bodily fluid. The method includes delivering a composition comprising at least one activity sensor into a subject, using a process in the body to deliver an active form of the activity sensor to a target site within the body, and detecting a signal such as a detectable reporter released from the activity sensor at the target site to thereby sense activity of the body at the target site. Preferably the process is a natural process of the body, e.g., that must occur for the activity sensor to present the reporter to enzymes at the target site in a cleavable form. The process may include collection of the activity sensor in the liver, cleavage of the activity sensor by one or more enzymes of the liver to release the detectable reporter into circulation, and glomerular filtration to release the detectable reporter in urine. The activity sensor may include the detectable reporter linked to a carrier, in which the carrier comprises one or more polymeric subunits.

Preferably, the composition includes a plurality of activity sensors, wherein each activity sensor includes a plurality of detectable reporters such that the composition releases the detectable reporters in the presence of a plurality of cognate enzymes. The enzymes may include one or more of the enzymes that exhibit elevated activity in association with a disease state. The enzymes may further include at least one enzyme not specific to the disease state. Optionally, each reporter comprises a polypeptide that is cleaved by a respective cognate enzyme, and wherein each reporter is linked to the carrier. The carrier may be, for example, a multi-arm polyethylene glycol scaffold to which each reporter is covalently linked (e.g., the carrier may have a molecular mass between about twenty and fifty kDa, and each distinct polypeptide that is cleaved may have a characteristic mass-to-charge ratio).

In some embodiments, the activity sensor comprises the detectable reporter linked to a carrier and is linked to an antibody for a tumor antigen. The process may include collection of the activity sensor at a tumor by means of interaction between the antibody and the antigen.

In certain embodiments, the activity sensor includes a therapeutic and the process releases the therapeutic at the target site. The composition may include activity sensors designed to show activities that result from the administration of the therapeutic. To illustrate, where the therapeutic includes a checkpoint inhibitor, the activity sensor may include substrates for tumor-secreted enzymes such as matrix metalloproteases. When the tumor is active, the enzymes cleave the activity sensors releasing peptide fragments as a signal detectable in urine. When the checkpoint inhibitor is effective, immune cells will begin destruction of the tumor cells, the secreted enzymes will decrease in quantity and activity level, and the signal will abate.

In some embodiments, the activity sensor comprises the detectable reporter linked to a carrier, further wherein the activity sensor is linked to a carbohydrate. The process may include binding of the carbohydrate to a cell-surface receptor on cells of a specific type and uptake of the activity sensor into the cells of the specific type.

In certain embodiments, the activity sensor is bound to a molecular cap and the process includes delivering into the body an enzyme that cleaves the molecular cap and releases the active activity sensor.

In some aspects, the disclosure provides methods of detecting activity in an organism. The methods include introducing a pro-analyte into the organism; allowing the organism to process the pro-analyte in a site-specific manner to generate a signal; and measuring the signal to identify activity associated with a physiological state of interest in the organism. The activity may include enzymatic cleavage of the pro-analyte to release detectable reporters. Optionally, the detectable reporters include a plurality of peptide fragments released upon the cleavage by a respective plurality of enzymes that are present in a site under the state of interest.

The organism may be a human subject. In certain embodiments, methods and composition of the invention are used with non-human organisms and are useful in agriculture or research. For research application, the organism may be, for example, a tobacco plant (e.g., Nicotiana tabacum), Caenorhabditis elegans, Drosophila, a zebrafish (Danio rerio), a pig (Sus scrofa), a Xenopus frog, or a mouse (e.g., Mus musculus). In some agricultural embodiments, the organism is a crop plant such as corn, wheat, maize, rapeseed, soybean, sunflower, barley, sorghum, potato, or rice. In certain agricultural embodiments, the organism is a livestock animal (e.g., cattle, horse, goat, sheep, swine, and poultry).

Aspects of the disclosure provide compositions that include pro-analytes that, when acted upon by a process in a body, will deliver an active form of an activity sensor to a target site within the body. The activity sensor is susceptible to cleavage by one or more enzymes within the body to release a detectable reporter that can be detected in a sample from the body. The presence of the detectable reporter in the sample indicates activity of the one or more enzymes at the target site within the body. Preferably, the pro-analyte is designed to be acted upon by a natural process of the body that must occur for the activity sensor to present the reporter to enzymes at the target site in a cleavable form. For example, the pro-analyte may include an activity sensor with a functional domain that interacts with molecular species in the body. Such a functional domain may include, for example, an antibody or antigen, a lectin or carbohydrate, a ligand for a cell surface receptor, or a substrate for enzymatic or chemical cleavage within the body. The pro-analyte may further include a therapeutic such that the process releases the therapeutic at the target site.

In certain embodiments, the pro-analyte includes the activity sensor in which the detectable reporter is linked to a carrier. The carrier may include, for example, one or more polymeric subunits. For example, the carrier may be a multi-arm PEG scaffold (e.g., 8-arm PEG at about 40 kDa). Preferably, the composition includes a plurality of activity sensors, each of which includes a plurality of detectable reporters such that the composition releases the detectable reporters in the presence of a plurality of cognate enzymes. Optionally, each activity sensor has a number (e.g., 8) of substantially identical polypeptide side chains that, when cleaved by a protease, release a portion of the polypeptide side chain as the detectable reporter. The composition may release detectable reporters for a plurality of different enzymes by having numerous activity sensors, each with peptide side chains specific for one protease. Preferably, the composition releases the detectable reporters upon encountering a plurality of enzymes, which enzymes may include one or more enzymes that exhibit elevated activity in association with a disease state. Those enzymes may further include at least one enzyme not specific to the disease state. That is, the composition may include certain activity sensors as a “control”, or to report a baseline activity level for one or more enzymes not specifically associated with the disease.

In certain embodiments, each reporter comprises a polypeptide that is cleaved by its respective cognate enzyme, and each reporter is linked to the carrier. The carrier may include a multi-arm polyethylene glycol scaffold to which each reporter is covalently linked. For example, the carrier may have a mass between about twenty and fifty kDa. In some embodiments, each distinct polypeptide that is cleaved has a characteristic mass-to-charge ratio and products from the composition are detectable by mass spectrometry.

The composition is designed to take advantage of the body as sample prep. Molecular features of the compositions are designed such that a natural process of the body delivers an active form of the activity sensor to a specific site in tissue or bodily compartment. For example, in some embodiments, the carrier and reporters are designed to have a molecular mass such that the activity sensors collect in the liver. Use of an 8-arm, 40 kDa PEG scaffold with 8 polypeptide side chains, for example, allows the compositions to present protease substrates within the liver, which substrates are cleaved to release detectable reporters upon encountering proteases that are characteristic of liver disease. Thus, in some embodiments, the process includes collection of the activity sensor in the liver, cleavage of the activity sensor by one or more enzymes of the liver to release the detectable reporter into circulation, and glomerular filtration to release the detectable reporter in urine.

Other bodily processes may be employed for delivery of the activity sensors. For example, in some embodiments, each activity sensor comprises the detectable reporter linked to a carrier, and the activity sensor is also linked to an antibody for a tumor antigen. The process includes collection of the activity sensor at a tumor by means of interaction between the antibody and the antigen.

In certain embodiments, the activity sensor is linked to a carbohydrate. The process includes binding of the carbohydrate to a cell-surface receptor on cells of a specific type and uptake of the activity sensor into the cells of the specific type. In some embodiments, the activity sensor is bound to a molecular cap or blocking group. The polypeptide chains are protease substrates that would release the detectable reporter upon encountering the proteases, but for the blocking group sterically hindering access of the chains to the proteases. The composition is delivered into the body and the active form of the activity sensor is not presented at the target site or compartment until the molecular cap or blocking group is liberated. This embodiment can make use of enzymatic activity within the body to release the cap or blocking group, or a chemical environment of the body can liberate the cap or blocking group (e.g., the cap is released by acid-catalyzed hydrolysis only upon passage through the digestive system). Alternatively, an exogenous enzyme that liberates the cap or blocking group can be delivered such that the active form of the activity sensor is not present until both the composition and the exogenous enzyme have been delivered into the patient and interacted with each other.

Methods of the invention may further comprise providing a report based upon detected reporters that indicates the health status of the patient. The report may contain a diagnosis or other actionable information; or may simply reflect detectable reporters identified in the sample.

### BRIEF DESCRIPTION OF THE DRAWINGS

FIG. 1 diagrams steps of a method of preparing a sample for analysis.

FIG. 2 illustrates a composition useful in methods of the disclosure.

FIG. 3 gives a detail view of an activity sensor.

FIG. 4 shows a multi-arm PEG scaffold used in activity sensor.

FIG. 5 shows a size filtration process within the body.

FIG. 6 shows steps in determining a composition of to the disclosure.

FIG. 7 shows an assay result by which one may measure detectable reporters FIG. 8 shows a report as may be provided using methods of the disclosure.

FIG. 9 shows data from selecting enzymes that are characteristic of stage 2 NASH.

FIG. 10 shows classification accuracy over number of proteases.

FIG. 11 illustrates an activity sensor with detectable reporters and molecular ligands.

FIG. 12 illustrates an activity sensor.

FIG. 13 illustrates a carbohydrate-linked composition.

FIG. 14 shows a composition with blocking groups.

FIG. 15 shows a lipid-modified composition.

### DETAILED DESCRIPTION

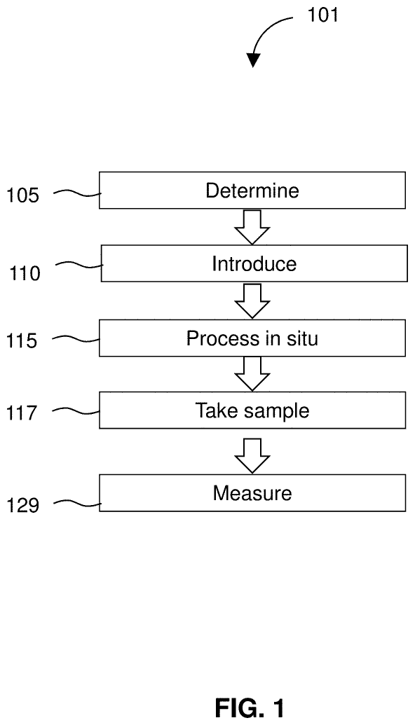

FIG. 1 diagrams steps of a method 101 of preparing a sample for analysis. The method uses the body as sample prep in that a pro-analyte activity sensor is used that is acted upon in the body to release a signal. Detection of that signal shows certain activities or combinations of activities at a site within the body. In some embodiments, the activity sensors are acted upon only by specific enzymes in the body. Activity by those enzymes may cleave reporters from the activity sensors such that a sample taken from the body includes those reporters. The activity sensors may be used to report any of a variety of activities within the body such as different competitive binding events, cleavage or conformational changes associated with chemical or mechanical stresses in the body, or modifications including glycosylation or phosphorylation activities sometimes associated with post-translational modification. By such actions, the body itself prepares the sample. Performing an assay on a sample from the body measures the various individual reports, which shows that certain activities occurred within the body. The pro-analyte is designed to include activity sensors for a plurality of enzymes or conditions that, take together, are indicative of a physiological condition or disease of interest.

Methods of the disclosure take advantage of the unique constellation of enzymes that are active in a disease state. That disease-specific set of enzymes will typically include enzymes that have increased activity at a site within the body affected by the disease, but the set may also include one or more enzymes that have decreased activity. The disease-specific set of enzymes will act on the activity sensors to release a detectable reporter. The identities and quantities of the detectable reporter provides a sensitive and accurate report on the presence or stage of a the particular disease or physiological state in the organ, site, or bodily compartment within the body. Thus the disclosure provides methods 101 that use the body as a sample prep vehicle.

The method 101 include determining 105 a set of enzymes that will exhibit disease-specific activity levels in a particular organ or site in tissue or a bodily compartment. A pro-analyte composition that includes activity sensors for those enzymes is made, and the method 101 includes introducing 110 the pro-analyte into a body of a patient. The body is allowed to process 115 the pro-analyte in situ in a site-specific manner to release a detectable reporter, and the method 101 includes taking 117 a sample from the body of the patient and measuring 129 the detectable reporter in the sample to identify a condition in the patient. Preferably, the pro-analyte is introduced 110 as a composition that includes a plurality of activity sensors.

The method 101 may also include determining activities or conditions to be tested for using activity sensors of the disclosure. Such activities or conditions include binding (e.g., competitive binding) events or conditions; cellular damage attributable to mechanical, metabolic, or chemical conditions; conformational changes to proteins associated with chemical, mechanical, or metabolic conditions; and protein modifications. For example, the activity sensors may probe for competitive binding by including peptide side chains that bind to a cognate receptor. In the presence of an excess of naturally-occurring ligand, the activity sensors do not bind and instead or available for cleavage by proteases. In another example, the activity sensors include peptide side chains that assume different conformations depending upon conditions within the body. In a first conformation, the peptides are cleaved to release a reporter and in a second conformation, the peptides are not cleaved. Detection of the reporter thus reports on local conditions within the tissue. Similarly, the activity sensors may probe for certain hallmarks of post-translational modifications. For example, if the activity sensors are extensively glycosylated or phosphorylated, they may be rendered unavailable for cleavage to release the detectable reporter, such that detection of the reporter signal can show a level of such modification activity.

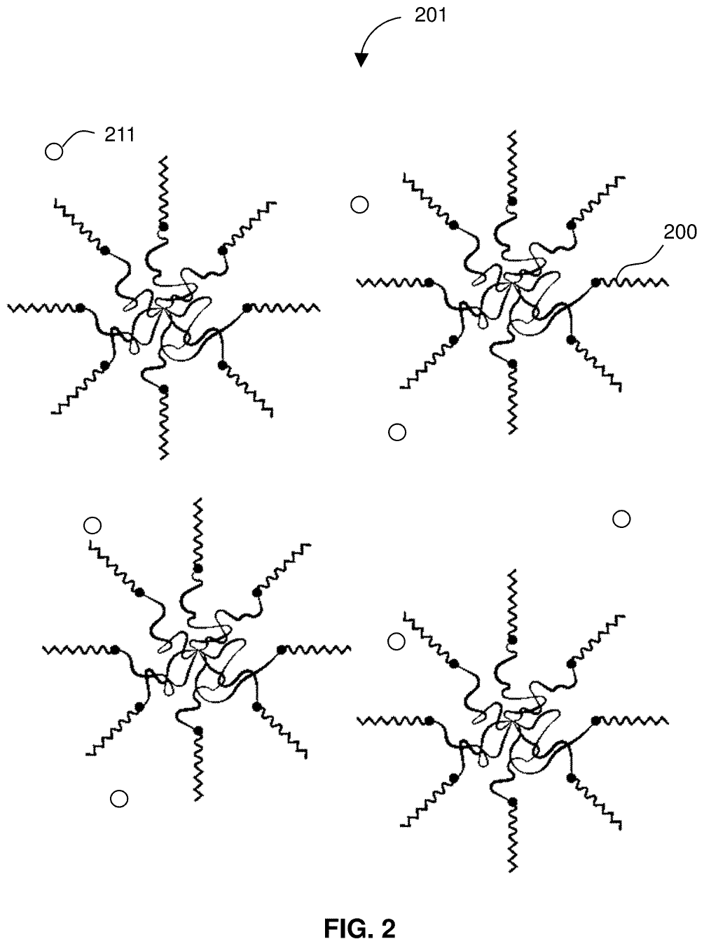

FIG. 2 illustrates a composition 201 useful in methods of the disclosure. The composition includes a plurality of activity sensors 200. Preferably, the composition includes a plurality of activity sensors, each of which includes a plurality of detectable reporters such that the composition releases the detectable reporters in the presence of a plurality of cognate enzymes. Optionally, each activity sensor has a number (e.g., 8) of substantially identical polypeptides that, when cleaved by a protease, release peptide fragments as the detectable reporter. The composition may release detectable reporters for a plurality of different enzymes by having numerous activity sensors 200, each with polypeptides specific for one protease. Preferably, the composition 201 releases the detectable reporters upon encounter a plurality of enzymes, which enzymes may include one or more enzymes that exhibit elevated activity in association with a disease state. Those enzymes may further include at least one enzyme not specific to the diseases state. That is, the composition may include certain activity sensors as a “control”, or to report a baseline activity level for one or more enzymes not specifically associated with the disease. The composition 201 also optionally includes a suitable carrier 211, such as a pharmaceutically acceptable solution or gel.

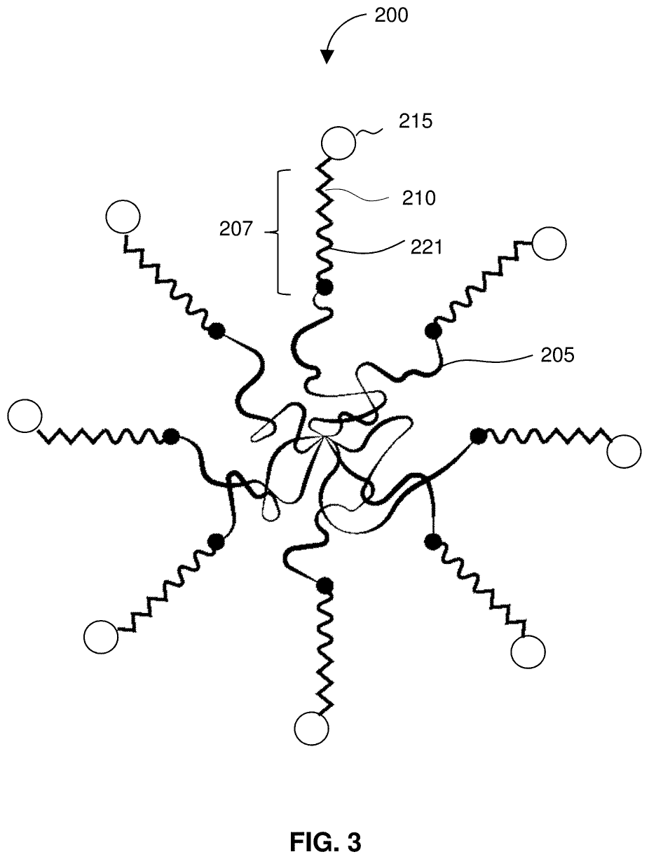

FIG. 3 gives a detail view of an activity sensor 200 according to embodiments of the disclosure. Each activity sensor 200 includes at least one detectable reporter 210. The detectable reporter may be linked to a carrier 205. The detectable reporter may be provided as polypeptide 207 that may be, for example, linked to a carrier 205. The polypeptide 207 preferably includes a cleavage site 221, e.g., a scissile bond susceptible to cleavage by a protease. The activity sensor 200 optionally includes a pro-analyte moiety 215 that is removed by a process in the body. Preferably, the activity sensor 200 includes a plurality of detectable reporters 210 such that the detectable reporters are released as peptide fragments in the presence of an enzyme that cleaves the polypeptide 207 at the cleavage site 221. Where the cleavage site 221 is specific to an enzyme that exhibits elevated activity in association with a disease state, the activity sensor 200 releases, into a sample that can be taken from the patient, the detectable reporter 210. Detection of the reporter in the sample is indicative of activity of the enzyme and thus indicative of disease. As discussed above, a composition 201 may include a plurality of the sensors 200; each sensor 200 may include a plurality of the detectable reporter 210; and each reporter may include a polypeptide 207 that is cleaved by its respective cognate enzyme to release a peptide fragment. Optionally, each reporter 210 is linked to a carrier 205. The carrier 205 may optionally be made up of one more polymeric subunits such as, for example, one a complex of any suitable biocompatible polymer including, for example, polyethylene glycol, lipids, polylactic acid, a multi-unit carbohydrate, peptidoglycan, agarose, cellulose, others, or combinations thereof.

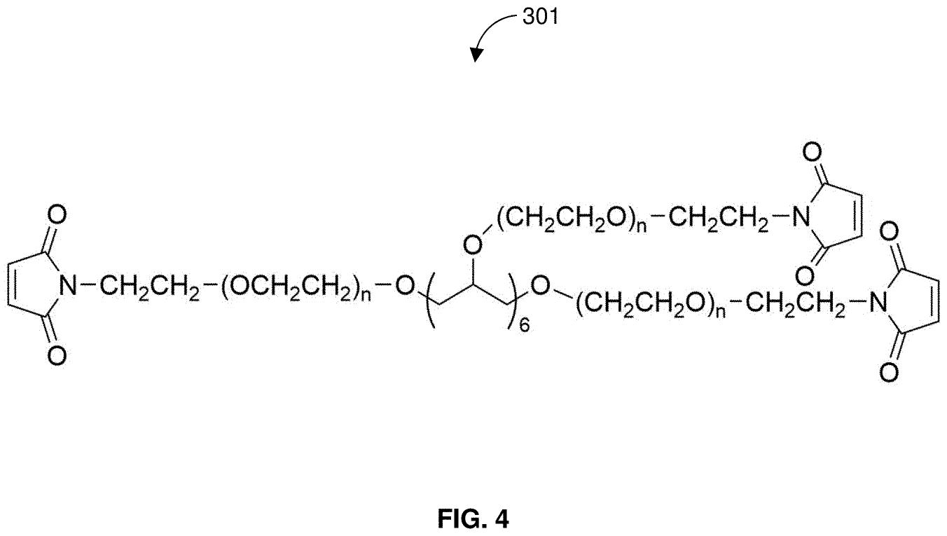

FIG. 4 illustrates a carrier 301 useful in certain embodiments. In some embodiments, the carrier comprises a polymer with a mass between about twenty and fifty kDa. The carrier may include a multi-arm polyethylene glycol (PEG) scaffold to which each reporter is covalently linked. The depicted carrier includes an 8-arm, 40 kDa PEG scaffold. Thus in certain embodiments, the disclosure provides a pro-analyte that includes an activity sensor in which the detectable reporter is linked to a carrier comprising, for example, a one more polymeric subunits. For example, the carrier may be a multi-arm PEG scaffold (e.g., 8-arm PEG at about 40 kDa).

Preferably, the composition 201 includes a plurality of activity sensors 200, each of which includes a plurality of detectable reporters such that the composition releases the detectable reporters as peptide fragments in the presence of a plurality of cognate enzymes. Optionally, each activity sensor 200 has a number (e.g., 8) of substantially identical polypeptide side chains that, when cleaved by a protease, release the peptide fragment as the detectable reporter. The composition may release detectable reporters for a plurality of different enzymes by having numerous activity sensors, each with peptide side chains specific for one protease. Preferably, the composition releases the detectable reporters upon encountering a plurality of enzymes, which enzymes may include one or more enzymes that exhibit elevated activity in association with a disease state. Those enzymes may further include at least one enzyme not specific to the diseases state. That is, the composition may include certain activity sensors as a “control”, or to report a baseline activity level for one or more enzymes not specifically associated with the disease.

The activity sensors are useful to probe the physiological state of tissue or a bodily fluid within the body. Numerous physiological states of interest such as various specific diseases are characterized by the expression and activity of certain enzymes. In general, the invention provides methods of detecting activity within a body. The methods include introducing a pro-analyte into a body of a patient; allowing the body to process the pro-analyte in a site-specific manner to give a signal; and detecting the signal to identify one or more activity within the patient. The signal may include any detectable signal that is provided when the body interacts with the pro-analyte. For example, processing the pro-analyte within the body may emit a characteristic change in a nuclease-magnetic resonance reading or cause a measurable change in heart rate of the patient. The signal may include compounds or molecules detectable by, for example, chromatography of the patient's breath, sweat, or blood. For example, an inhaled pro-analyte may release volatile organic compounds (VOCs) under certain activity within the lungs, such that the VOCs are detectable via breathalyzer test. The method may include taking a sample from the body of the patient and measuring the signal in the sample to identify the activity in the patient.

In certain embodiments of the methods the signal includes the release of a detectable reporter from the pro-analyte. The detectable reporters may include a plurality of peptide fragments released upon cleavage of the pro-analyte by a respective plurality of enzymes that are present in a site within the body under the condition (e.g., such that measuring the detectable reporters includes quantifying the peptide fragments in the sample). The body may process the pro-analyte by means of a plurality of enzymes that are unique to the condition (e.g., by enzymatic cleavage to release the detectable reporters).

The body processes the pro-analyte by enzymatic cleavage to release the detectable reporter and the detectable reporter may be a plurality of peptide fragments detectable by an assay of the sample. Any suitable sample may be taken from the body and assayed for the reporter. For example, the sample may include breath, urine, blood, plasma, serum, lymph, saliva, stool, sputum, sweat, hair, or any other suitable material. In a preferred embodiment, the sample is a urine sample. The sample may be assayed by any suitable method or technique for the reporter. Suitable assays include mass spectrometry, electrophoresis, immune-assays, fluorescent probes, others, and combinations thereof.

In some embodiments, the pro-analyte gives a signal indicative of other actions or interactions within the body. For example, the pro-analyte may give a signal that shows an activity that results from something caused by administration of the pro-analyte itself. Giving the pro-analyte may induce an immune response, one or more binding events, clotting or other cross-linking events, or steric or chemical responses to the pro-analyte. In some embodiments, the pro-analyte is used to report competitive binding events. For example, noting that a signal from the activity sensors gives a rate of an activity, enzyme kinetics may be inferred and competitive binding of other (e.g., naturally-occurring) substrates may be detected across a number of measurements. The methods may be used to detect activity associated with protein modifications. For example, glycation events or phosphorylation of a reporter (or inducing phosphorylation, etc. of an endogenous protein/molecule) can be detected directly in the sample or inferred where, for example, modification renders the activity sensors more or less susceptible to the activity. Secondary interactions may be used to provide a readout of certain conditions such as the effect of a certain drug on the cells. For example, the activity that goes into killing a cell (signal A) and the signal that results from the cell being killed (signal B) may happen in close proximity to each other (giving signal A+signal B), which is a different signal than if A and B are spatially distinct. In certain embodiments, the activity sensors are cleaved within the body by enzymes to release, as the signal, detectable reporters such as peptide fragments.

When those enzymes cleave the activity sensors to release the detectable reporters, detection of those reporters in samples from the body indicates the presence of the physiological state of the tissue. Of note, many diseases involve mechanisms of action by which enzyme activity is dysregulated prior to the appearance of other symptoms. For example, neoplastic cells will release extracellular tissue remodeling enzymes to cleave the extracellular matrix before the cells proliferate into a detectable tumor and before those tumor cells release a level of tumor DNA fragments that can be detectable by liquid biopsy. Accordingly, the precise detection of extracellular proteases at a specific site is indicative of an important physiological state.

The disclosure provides analytical methods that use the body for sample preparation steps to provide a sample that includes readily-detectable disease markers. A pro-analyte composition is designed and made or obtained, and the pro-analyte composition is administered to a subject. The body is then allowed to process the pro-analyte in situ, in a site-specific manner. A sample taken from the body includes a detectable reporter released from the pro-analyte. The detectable reporter includes the products of enzymatic processing of the pro-analyte at the affected site within the body.

Methods of the disclosure include determining 105 the composition of the pro-analyte so that the pro-analyte present substrates for the disease-specific panel of enzymes at the affected tissue or bodily compartment. The bodily processing is site-specific in that those enzymes will not exhibit their disease-characteristic activity levels across the enzymes in the represented groups at locations other than the affected tissue or site. For example, if the probed disease involves nonalcoholic steatohepatitis (NASH), the pro-analyte will include substrates for a plurality of proteases that have been shown to be differentially expressed in NASH. Thus the composition may include activity sensors that are specific for several of (e.g., 5, or 8, or 10, or 12 of) FAP, MMP2, ADAMTS2, FURIN, MMP14, MMP8, MMP11, CTSD, CTSA, MMP12, MMP9, and ST14. Only upon entering a liver affected by NASH will the pro-analyte be processed in situ, in a site-specific manner within the body to release the corresponding substrates as a detectable reporter in a sample obtainable from the body.

Additionally, as described below, certain embodiments of the disclosure further use a process in the body to deliver an active form of the activity sensor to a target site within the body. Any suitable bodily process may be exploited to aid in delivery of the active form of the activity sensor. For example, blocking groups may be cleaved off, the activity sensor may bind to certain cells or molecules, or the pro-analyte form may be acted upon by enzymes, either endogenous or exogenous, within the body to transform it into a an active form of the activity sensor. In certain embodiments, the pro-analyte is subject to one or more steps of filtration with in the body.

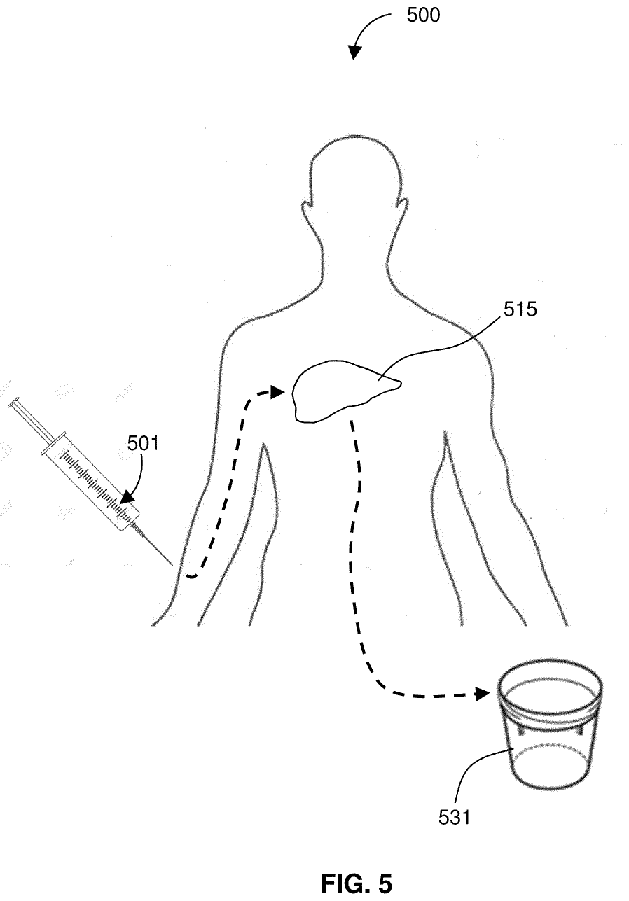

FIG. 5 shows a size filtration process within the body that aids in collecting activity sensors within the liver 515, and again in collecting detectable reporters in urine 531. In the depicted embodiments, a pro-analyte 501 that includes activity sensors is delivered into a subject. The activity sensors include detectable reporters and are designed to have a molecular mass such that the activity sensors collect in the liver. Use of an 8-arm, 40 kDa PEG scaffold with 8 polypeptide side chains, for example, allows the composition to present protease substrates within the liver.

The body is allowed to process 115 the pro-analyte 501 in situ. In that processing, a set of disease-associated enzymes cleave the activity sensors to release peptide fragments as a detectable reporter. After cleavage of the activity sensor by one or more enzymes of the liver to release the detectable reporter 210 into circulation, glomerular filtration releases the detectable reporter in urine 531. Preliminary results indicate that detection of the detectable reporters 210 in the urine 531 may take place within less than a few hours after delivery of the composition 501 into the patient 500. Due to the processing by the body, the sample taken 117 from the body may be directly assayed (e.g., by mass spectrometry, gel electrophoresis, an immuno-assay, or other such suitable assay) to measure the detectable reporter and accurately report the presence and/or stage of the disease.

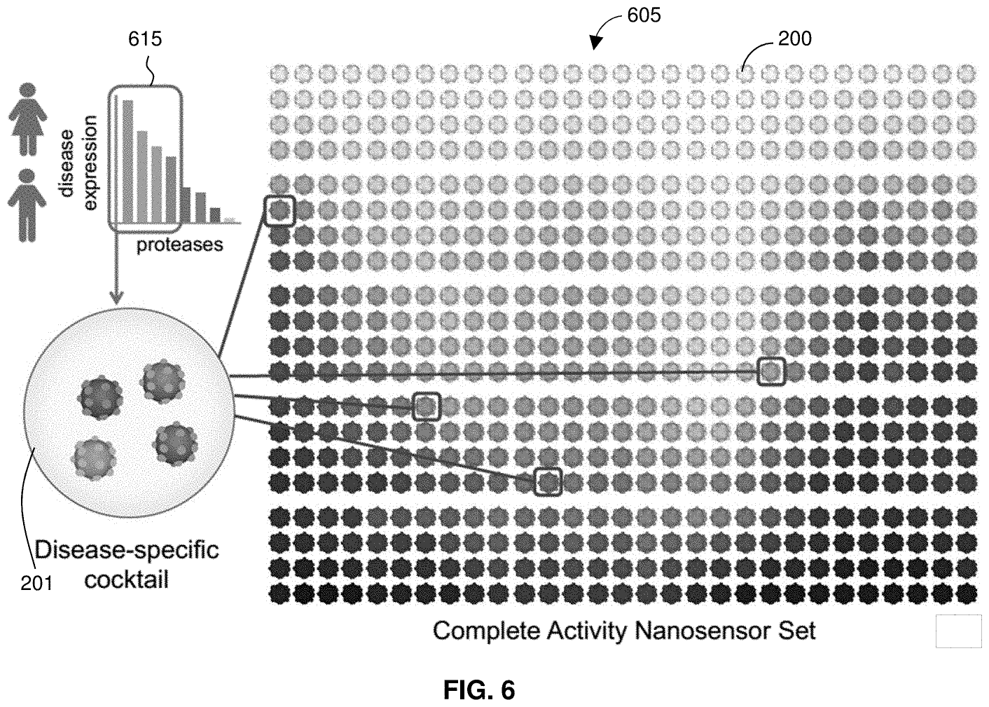

FIG. 6 illustrates determining a composition 201 according to the disclosure. Preferably, a composition 201 is made by drawing from a library 605 that contains a complete set of activity sensors 200. In certain embodiments, each activity sensor 200 within the library 605 includes a plurality of identical polypeptides 207.

Each polypeptide 207 will, upon cleavage by processing within the body, release a peptide fragment. In preferred embodiments, the detectable reporter in the sample includes a plurality of peptide fragments released upon cleavage of the pro-analyte by a respective plurality of enzymes (corresponding to members of library 605) that are present in a site within the body under the condition. Preferably, measuring the detectable reporter includes quantifying the peptide fragments in the sample. This may be done, for example, by measuring a peak height in a mass spectrum. The composition preferably includes a plurality of members of the library 605 selected to be specific to a disease or condition of interest such that the body processes the pro-analyte by means of a plurality of enzymes that are unique to the condition. The body processes the pro-analyte by enzymatic cleavage to release the peptide fragments that make up the detectable reporter. The detectable reporter comprises a plurality of peptide fragments detectable by an assay of the sample.

Each entry in library 605 may be represented by an arbitrary number (e.g., thousands) of copies that may be identical or essentially identical. As shown, the library 605 has 580 unique activity sensors 200. Each one may be present in thousands of copies stored in a suitable container. For example, in some embodiments, the hundreds or thousands of copies of each library member is stored in its own container such as a centrifuge tube such as the 1.5 mL micro-centrifuge tube sold under the trademark EPPENDORF FLEX-TUBES by Eppendorf, Inc. (Enfield, Conn.). For any given biological or physiological condition of interest, a profile 615 of relevant proteases is developed or obtained.

One may obtain a profile 615 of those enzymes that are differentially expressed under the physiological state of interest, and such enzymes are preferably included in the profile 615. However, the profile 615 may also include other enzymes, such as one or more control enzymes that are assumed to be constitutively expressed and active in the tissue or bodily compartment of interest. The control enzyme may give a background activity level against which to calibrate or normalize the activity levels of the disease-associated enzymes. Additionally, the profile may include one or more enzymes that are understood to be specific for a frequent or likely comorbidity of the physiological state of interest. In one example, it is understood that liver diseases (e.g., nonalcoholic fatty liver disease (NAFLD), nonalcoholic steatohepatitis (NASH), hepatocellular Carcinoma, and cirrhosis) are frequently associated with obesity. Thus where the physiological state if interest is a liver disease or a stage of a liver disease, the profile 615 may include enzymes that are known to be specifically and differentially expressed in connection with obesity. The profile 615 may also include enzymes that are differentially expressed under other significant conditions such as pregnancy or within the bodies of smokers.

The activity sensors 200 specific to the enzymes in the profile 615 are composed together to form a composition 201 of the disclosure. Thus methods 101 provide for designing 105 compositions 201 that include activity sensors 200 in which each activity sensor includes a plurality of detectable reporters such that the composition releases the detectable reporters in the presence of a plurality of cognate enzymes. When the body is allowed to process the pro-analyte, the in situ processing releases the detectable reporter in quantities measurably different than quantities at which the reporter would be released elsewhere in the body or in the absence of the disease. In most preferred embodiments, the pro-analyte is administered as a composition comprising a plurality of activity sensors that release the detectable reporter in the presence of a plurality of cognate enzymes. The detectable reporter includes a plurality of distinct peptide fragments. Each activity sensor preferably includes a plurality of polypeptides that are cleaved by the enzymes to release the detectable reporter. Preferably, the enzymes include one or more of the enzymes that exhibit elevated activity in association with a disease state, however, it may be beneficial for the enzymes to also include at least one enzyme not specific to the diseases state. Those enzymes include one or more of the enzymes that exhibit elevated activity in association with a disease state. In preferred embodiments, the enzymes also include at least one enzyme not specific to the disease state. Cleavage of the activity sensors by the enzymes releases detectable reporters into a bodily sample.

Any suitable sample may be taken and assayed for the detectable reporters by any suitable assay. Suitable bodily samples, depending on the nature of localization of the sensors 200, include blood, urine, sweat, lymph, a biopsy, fine needle aspirate, exhaled breath, a swab (e.g., cheek swab), tears, mucous, cerebrospinal fluid, tissue sample, resected tumor sample, hair or nail clipping, cartilage excision, synovial fluid (e.g., from a joint affected or suspected to be affected by osteoarthritis), or any other suitable sample known in the art. Suitable assays for detecting the detectable reporters include, for example, enzyme-linked immunosorbent assays, other immunoblotting assay, mass spectrometry, secondary ion mass spectrometry, gel electrophoresis, two-dimensional electrophoresis, chromatography, HPLC, bead capture and separation (e.g., using magnetic beads that bind to the detectable reporters), or any other assay. Where the analytes each have a unique mass by virtue of the design of the polypeptide sequence, mass spectrometry may be performed on the urine sample to reveal the presence or absence of mass spectra signifying the presence or absence of the physiological state within a patient.

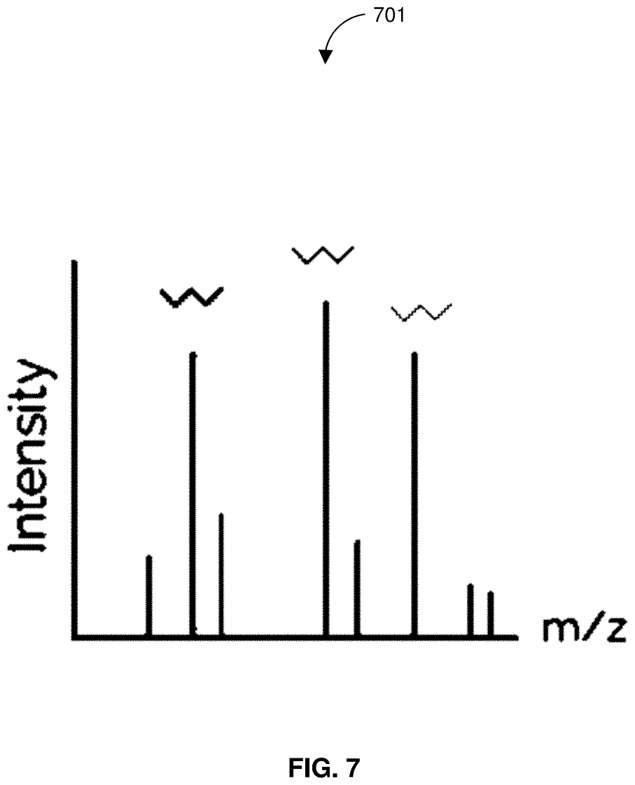

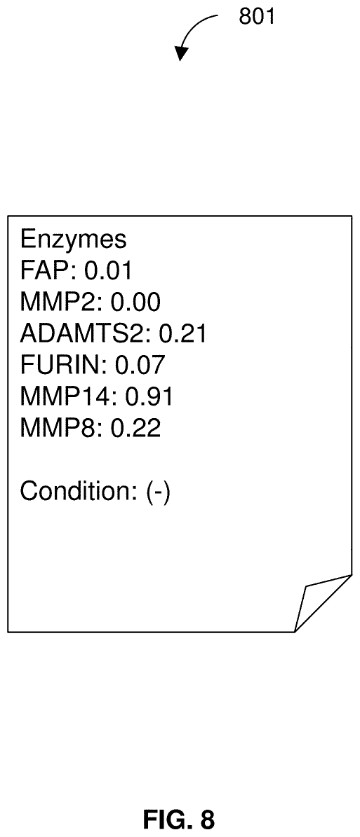

FIG. 7 shows an assay result 701 by which one may measure 129 the detectable reporters 210. In the depicted embodiment, the assay result 701 is a mass spectrum as may be obtained from a sample from a patient. The sawtooth lines represent peptide fragments released from the activity sensors 200, and that each has a distinguishing mass to charge (m/z) ratio. The presence of the indicated peaks on the mass spectra indicates that the proteases were present in the liver and cleaved the reporters.

In certain embodiments, an assay is performed that have a visible result, such as a paper strip as may be found on a dip stick with an ELISA or lateral flow assay. Suitable assays for detecting the detectable reporters include, for example, enzyme-linked immunosorbent assays, or other immunoblotting assay. E.g., where the sample is urine, a dipstick with a paper strip may be provided that undergoes a color change in the presence of detectable reporter(s). Such a color change may further be analyzed via imaging, e.g., by computer analysis of an image result from such an assay. Thus methods of the disclosure may include administering a pro-analyte to a patient, wherein in situ processing in the body results in enrichment of detectable reporters from the pro-analyte in proportion to a health condition of the patient. The detectable reporters may then be measured in a sample from the patient after which the measured reporters are correlated to the health condition in the patient. In some embodiments, measuring the detectable reporters comprises imaging the sample, and analyzing an image obtaining in the imaging step (e.g., with a computer). In certain embodiments, the measuring step further comprises distributing the sample on a strip of an ELISA or lateral-flow assay, using a mobile device to image the strip, and using an app on the mobile device to perform the analyzing step. Thus methods of the invention may be performed using a composition that includes the pro-analyte, an ex vivo detection assay such as an ELISA test, dipstick, or lateral flow assay, and image analysis tools such as a smartphone app. A smartphone with an app may be used to take a picture of the ex vivo detection assay result and analyze the image to provide the report.

Any suitable disease or physiological state may be activity-mapped according to the methods including, for example, cancer, osteoarthritis, infection by a pathogen, liver disease, others, and combinations thereof. Detecting 129 the detectable reporter in the sample indicates that the determined enzymes are active at the target site. Preferably, the method 101 includes providing a report that indicates activity or activity levels of one or more enzymes at the target site or indicates a physiological state or medical condition of interest in the tissue or bodily compartment of the patient.

FIG. 8 shows a report 801 as may be provided using methods 101 of the disclosure. Preferably, results of a readout 701 are quantified automatically using a computer. The results in the readout may be read into a patient file or database. The computer may be used to compose the patient results into a report 801 and the report may be provided as a paper output, digital file, or display.

Compositions 201 of the disclosure preferably include a plurality of activity sensors 200 that are specific to a physiological state of interest and in which the set of activity sensors may also include one more control activity sensors or sensors for comorbidities. The disclosure includes methods of the determining activity sensors 200 to be included in a composition. Methods of determining the sensors include determining what set of enzymes is differentially expressed under a given physiological state of interest and may further include identifying a representative subset of those differentially expressed enzymes wherein the representative subset is statistically supported as characteristic of the physiological state of interest.

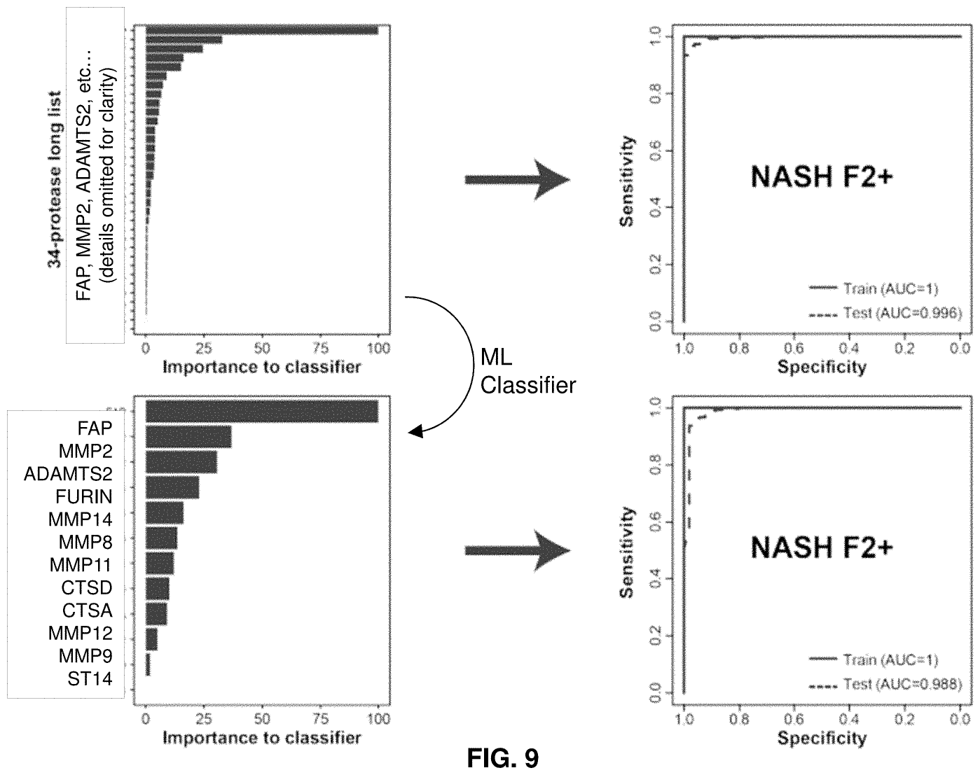

FIG. 9 shows data from selecting enzymes that are characteristic of stage 2 NASH. In one set of experiments, tissue known to be affected by stage 2 NASH was subject to expression profiling by RNA-Seq as was a control tissue. The resulting coding sequences were searched against databases (e.g., BLAST against GenBank) and all extracellular proteases differentially expressed under NASH stage 2 were identified. That list included 34 proteases: FAP, MMP2, ADAMTS2, FURIN, MMP14, GZMB, PRSS8, MMP8, ADAM12, CTSS, CTSA, CTSZ, CASP1, ADAMTS12, CTSD, CTSW, MMP11, MMP12, GZMA, MMP23B, MMP28, MMP7, ST14, MMP9, MMP15, ADAMDEC1, ADAMTS1, GZMK, KLK11, MMP19, PAPPA, CTSE, PCSKS, and PLAU. From the complete list, one may identify a subset of enzymes the presence of which discriminates for presence of the physiological condition. Any suitable method may be used to select the subset including, for example, simply taking the top several (e.g., top 8, top 10, top 12). Other methods include bootstrap sampling from the full list and validating the sampled list against results from the full list. In certain embodiments, a machine learning (ML) classifier is built which is trained on training data from subjects with known outcomes. The ML classifier is used to a select a subset of enzymes.

As shown in FIG. 9, when the full list of 34 proteases is used to assemble a composition 201 that includes a corresponding 34 unique activity sensors 200 (each present in hundreds or thousands of copies), that composition is useful to show the presence of NASH stage 2 with high sensitivity as shown by an area under the curve (AUC) of 0.996. A machine learning classifier selects a subset of the proteases that identify the disease with a threshold sensitivity and specificity, in which the subset is small enough that a corresponding set of protease substrates may be assembled into activity sensors 200 of a composition 201 that, when administered to a patient, are cleaved in the disease tissue to release detectable analytes signifying presence of the disease. Here, the ML classifier identified 12 proteases (FAP, MMP2, ADAMTS2, FURIN, MMP14, MMP8, MMP11, CTSD, CTSA, MMP12, MMP9, and ST14). When a composition that included those proteases was administered to a patient, a sample from the patient was assayed for the corresponding detectable reporters 210. Presence of the detectable reporters was correlated to the a positive identification of NASH stage 2. Even using the smaller subset of protease substrates, the composition was useful to identify the disease with great specificity giving an AUC of 0.998). Replicate statistical studies reveal a relationship between classification accuracy and a number of proteases included in a composition 201.

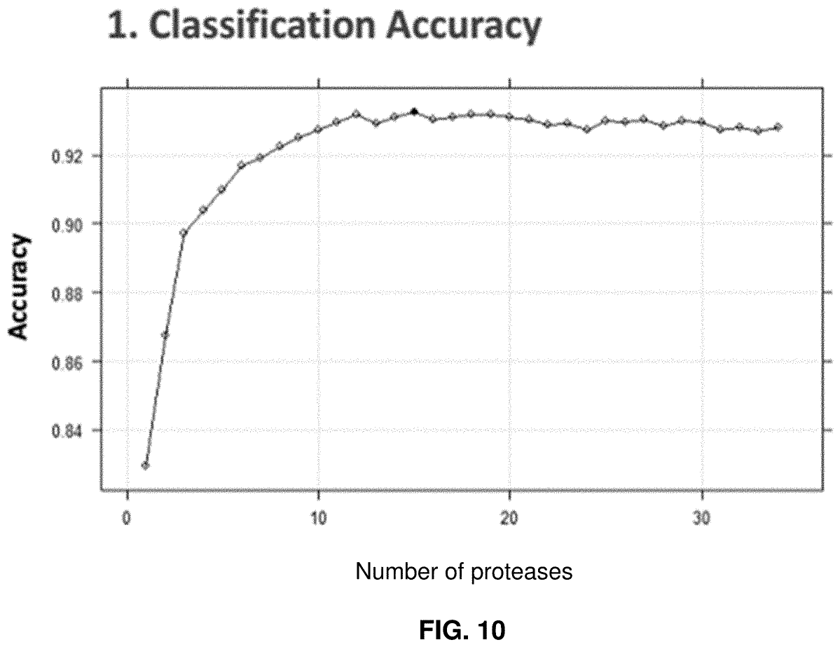

FIG. 10 shows classification accuracy over number of proteases included in a composition 201. That evidence tends to show that accuracy stabilizes as the number of proteases reaches about 10. Methods of the disclosure provide analytical methods for mapping activity in a disease-specific manner. Any of a variety of diseases or medical conditions may be mapped using methods and compositions of the disclosure. In preferred embodiments, proteases that are active in disease tissue and subject to differential expression relative to normal tissue are identified and activity sensors 200 are assembled in which the activity sensors include cleavage substrates for those proteases. For any given protease of interest, one of skill in the art is able to determine a substrate that will be cleaved by that protease. One may determine a protease's cognate substrate from the academic literature or from scientific research databases. For example, the protein databank (PDB) includes entries for most or all proteins of significance, and the PDB entries include a link to a full report that gives a substrate sequence for proteases. In one illustrious example, the matrix metalloproteinase 9 (MMP9) is entry number 4JIJ of the PDB and that entry includes the substrate sequence. A composition 201 of the disclosure is preferably designed to probe for activity of about 10 (e.g., 7, 8, 9, 10, 11, 12, or more) proteases and can be made by selecting activity sensors 200 from a library 1105. Each activity sensor 200 in the library 1105 may be specific to a protease and may be made by linking the cognate substrate to a scaffold such as one or more biocompatible macromolecules such as PEG.

The disclosure provides compositions and methods for efficient and specific delivery of targeted activity sensors to specific sites or compartments within the body. Activity sensors of the disclosures may be delivered in pro-analyte form and subject to processing by the body. A process within the body aids in presenting an active form of the activity sensor to a specific site within tissue or a bodily compartment such as blood or lymph. The administered composition, i.e., the pro-analyte, may be susceptible to processing within the body by any one or number of bodily processes including, for example, size filtration, enzymatic or chemical cleavage to remove blocking groups, receptor binding, or cellular uptake by for example the endocytic pathway. Embodiments of the disclosure employ the body for sample preparation. A composition may be delivered into the body whereupon a natural bodily process acts upon or changes a part of the composition so that the composition is active at an intended location.

Further features and embodiments are within the scope of the disclosure. For example, some aspects of the disclosure provide a method that uses a process in the body to deliver an active form of the activity sensor to a target site within the body. The method may include determining a target site within tissue or bodily compartment to be queried and one or more enzymes that, if active at the target site are indicative of a condition or physiological state of interest, such as a disease. The method includes delivering a composition into a body of a subject. The composition preferably includes at least one activity sensor that when acted upon by the determined enzymes release (e.g., produce as substrates of the enzymes) one or more detectable reporters. A process in the body is used to present an active form of the activity sensor to the target site. A sample may be taken from the body such as an exhaled breath sample, a urine sample, or a sample of sweat, blood, or tissue. Detecting the detectable reporter in the sample indicates that the determined enzymes are active at the target site. Preferably, the method includes providing a report that indicates activity or activity levels of one or more enzymes at the target site or indicates a physiological state or medical condition of interest in the tissue or bodily compartment of the patient. The activity sensors, which release detectable reporters when acted on by certain enzymes within the body, are provided as pro-analytes. Processes within the body deliver the activity sensors in active form to a target site of interest. The process may be a natural process of the body that must occur for the activity sensor to present the reporter to enzymes at the target site in a cleavable form.

By delivering the activity sensors in a pro-analyte form and taking advantage of the “body as sample prep” to locate the active form of the activity sensors at the target site, methods of the invention avoid premature or off-target cleavage of the activity sensors and release of the detectable reporter. Because off-target release of the detectable reporter is avoided, the reporters are only detected when acted upon by enzymes at the target site. Methods of the disclosure are useful for determining a physiological state or condition of a site within a subject when the state or condition is characterized by the activity of certain enzymes. For example, tumors are known to excrete proteases into the surrounding tissue in order to grow. Similarly, various etiologies of liver disease are characterized by heighted levels of protease activity in the liver as disease advances through stages. In another example, the onset of osteoarthritis is marked by the heightened protease activity in the affected joint. In common among these examples is that a composition of the disclosure may be used to specifically show and characterize the state of interest, i.e., the emergence of a tumor, a stage of liver disease, or the onset of osteoarthritis.

Other processes occurring within the body may be used to collect and deliver the activity sensors to a site of interest. For example, within the body, a composition of the disclosure may undergo enzymatic or chemical cleavage to remove blocking groups, binding to specific receptors or molecules, or cellular uptake by for example the endocytic pathway.

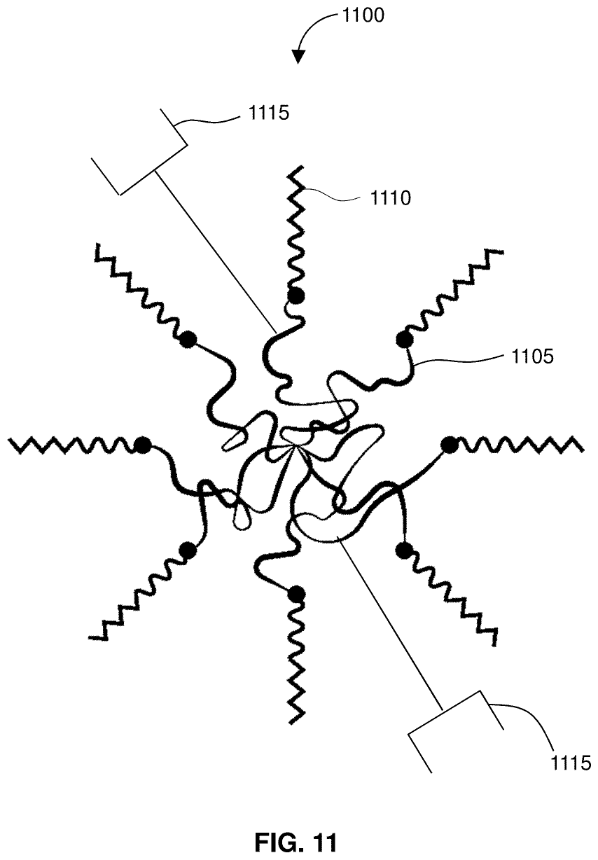

FIG. 11 illustrates an activity sensor useful in methods of the disclosure. The activity sensor includes a plurality of detectable reporters 1110, optionally linked to a carrier 1105, such as a molecular scaffold (e.g., one or more biocompatible molecular subunits such as PEG). Additionally, the activity sensor is modified to include one or a plurality of molecular ligands 1115 that bind to cognate receptor molecules within the body. Any suitable ligand 1115 may be present, including for example the ligand for a G protein coupled receptor (GPCR) or for a receptor tyrosine kinase such as a growth factor. Binding to a cognate receptor can localize the activity sensor at a particular cell type of interest, such as at and near the surface of neurons.

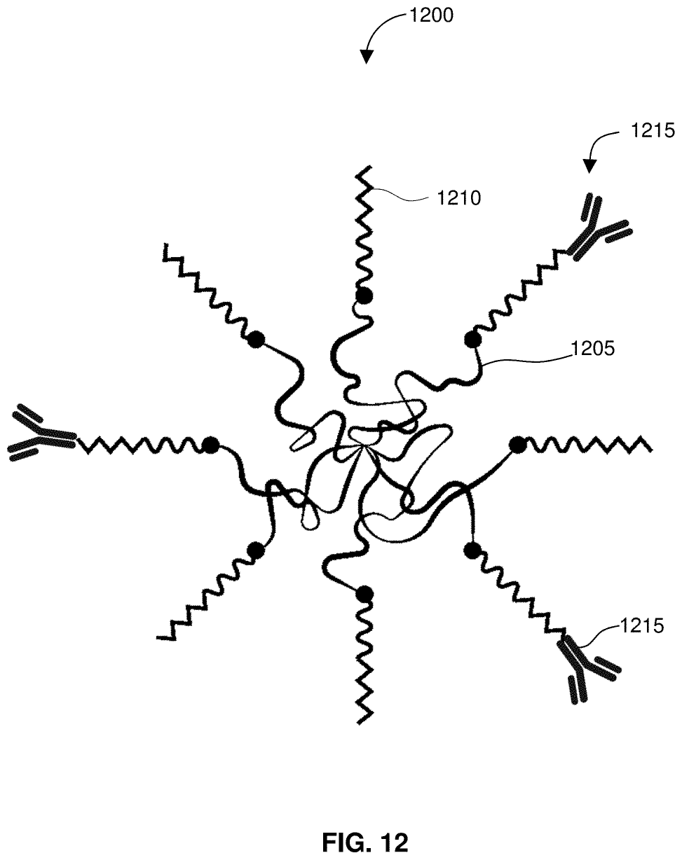

FIG. 12 illustrates an activity sensor 1200 useful in methods of the disclosure. In the depicted embodiment, the activity sensor 1200 includes a detectable reporter 1210 linked to a carrier 1205 and the activity sensor 1200 is also linked to an antibody 1215. When the activity sensor 1200 encounters an antigen for the antibody 1215, the activity sensor 1200 will effectively bind to the antigen. When the antigen is a cell-surface protein on a cell, the activity sensor will effectively bind to the cell. Methods are known in the art by which antibodies may be raised against antigens and antibodies with anti-tumor indications are known in the art. The activity sensor 1200 may include as antibody 1215 a commercially available antibody such as Nimotuzumab, Cetuximab, Panitumumab, or Trastuzumab for solid tumors or Rituximab, Ibritumomab tiuxetan, Ofatumumab, Obinutuzumab, Brentuximab vedotin, Mogamulizumab, or Blinatumomab for other malignancies. The antibody 1215 may be an antibody raised against a particular tumor, cell, tumor cell, or antigen. Antibodies can be raised by phage display/phage selection, by the “shadow-stick selection” process of Sorenson, 2011, Nature Protocols 6:509-522, which is incorporated by reference, or by other methods known in the art. See Sanches-Martin, 2017, Selection strategies for anti-cancer antibody discovery, Trends Biotechnol 33(5):292-301, incorporated by reference.

Inclusion of the antibody 1215 will localize the activity sensor 1200 to a tumor when the antibody binds tumor antigen. The process includes collection of the activity sensor at a tumor by means of interaction between the antibody 1215 and the antigen. When a composition of the disclosure includes activity sensors 1200 bearing antibodies 1215, the composition provides a sensitive reporter of tumor activity and methods of the disclosure provide for localizing an active form of the activity sensor to the tumor by means of antibody/tumor interaction. A sample may be taken from the body (e.g., the form of a fine needle aspirate from the tumor) and a quantity of cleaved detectable reporter 1210 present in the sample reports on the activity-level of tumor-associated proteases.

It is understood that tumors grow and invade surrounding tissue by expressing and releasing extracellular proteases that degrade extracellular matrix proteins in the tumor microenvironment. This degredation allows the tumor to penetrate the tissue and expand. Without being bound by any mechanism, it may be theorized that extracellular protease activity is one of the earliest phenomenon exhibited by neoplastic cells and compositions of the disclosure may be used to reveal tumor-specific activity before other existing methods such as liquid biopsy are capable of showing any tumor-specific activity. Other embodiments are within the scope of the disclosure.

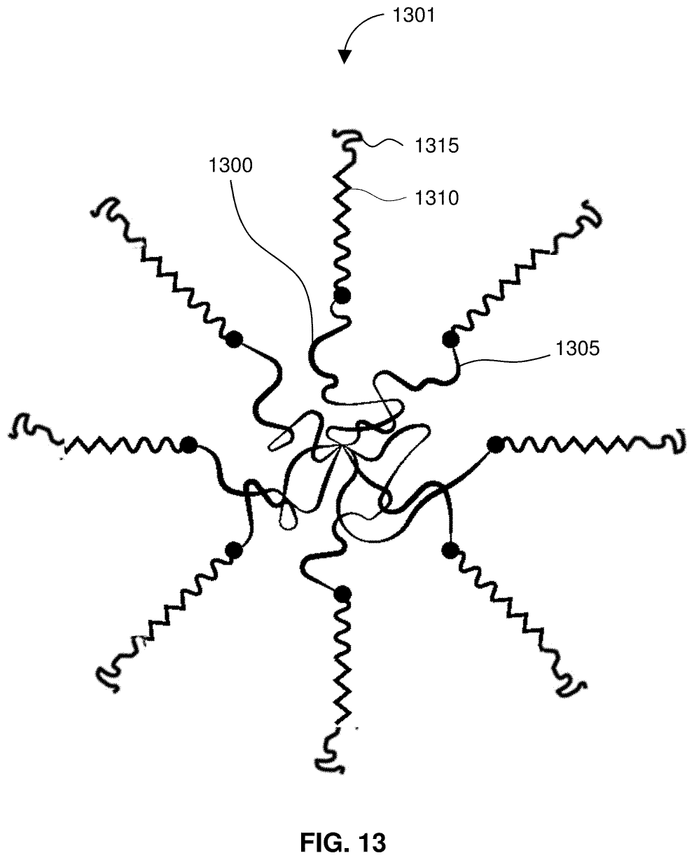

FIG. 13 illustrates a carbohydrate-linked composition 1301 useful in methods of the disclosure for inducing intracellular uptake of activity sensors. The composition 1301 preferably includes a plurality of activity sensors 1300 in which each activity sensor 1300 is linked to one or more carbohydrates 1315. In the depicted embodiment, the carbohydrates 1315 are linked to detectable reporters 1310. The detectable reporters 1310 may further be linked to a carrier 1305 such as a polymer scaffold. In the depicted embodiment, the process of method 101 includes binding of the carbohydrate 1315 to a cell-surface receptor on cells of a specific type and uptake of the activity sensor into the cells of the specific type. Cellular uptake by for example the endocytic pathway uses a principle uptake mechanism of cells for activity sensors 1300. Those sensors 1300 use carbohydrates 1315 to facilitate the endosomal escape and ensure cytosolic delivery of the activity sensors 1300. Different mechanisms such as pore formation in the endosomal membrane, pH-buffering and fusion into the lipid bilayer of endosomes have been proposed to facilitate the endosomal escape. Carbohydrates, which interact with some cell surface receptors, can serve as targeting ligands for activity sensors 1300. Carbohydrates permit glycol-targeting, which is based on endogenous lectin interactions with carbohydrates. Each activity sensor 1300 may include multiple interacting carbohydrates to achieve strong binding strength. One example uses galactose or galactose-mimics as ligands to asialoglycoprotein receptor, an endocytotic cell surface lectin receptor highly expressed on hepatocyte surfaces. DC-SIGN is a C-type lectin receptor preferentially expressed by dendritic cells. Lex and ManLAM carbohydrates, for example, can be used to enhance the binding and uptake of the activity sensors by dendritic cells. See Friedman, 2013, The smart targeting of nanoparticles, Curr Pharm Des 19(35):6315-6329, incorporated by reference. In some embodiments, instead of carbohydrates, the activity sensors include peptide side chains as cell-penetrating peptides.

Here, aspects of the disclosure provide a composition that includes a pro-analyte that, when acted upon by a process in a body, will deliver an active form of an activity sensor to a target site within the body. The activity sensor 1300 is susceptible to cleavage by one or more enzymes within the body to release a detectable reporter 1310 that can be detected in a sample from the body. The presence of the detectable reporter in the sample indicates activity of the one or more enzymes at the target site within the body. Preferably, the pro-analyte is designed to be acted upon by a natural process of the body that must occur for the activity sensor to present the reporter to enzymes at the target site in a cleavable form. For example, the pro-analyte may include an activity sensor 1300 with a functional domain 1315 that interacts with molecular species in the body. Such a functional domain may include, for example, a cell-penetrating peptide (CPP). Cell entry routes that contribute to the cytosolic translocation of CPP-activity sensor complexes include the direct cell membrane penetration and the endosomal pathway, requiring first the endocytic entry followed by endosomal escape. The occurrence of direct passage through the plasma membrane is understood to occur when CPPs alone are used.

The Pep-1 family peptides (also called Chariot peptides) can facilitate the uptake of an activity sensor into the cell also via a non-endocytic mechanism by forming a helical structure upon interaction with the plasma membrane lipids and inserting into the lipid bilayer. Also, Pep-1 can increase the uptake of cargo and indeed enhance its escape from the endocytic vesicles, yet the uptake mechanism of the peptide-protein complex on the plasma membrane follows the typical endocytic routes of macropinocytosis and clathrin-mediated endocytosis. It is therefore possible that some CPPs may promote the internalization of the cargo molecule through direct penetration across the plasma membrane while other CPPs attached to an activity sensor utilize the endocytic machinery to gain entry to cells. See Raagel, 2010, Peptide-mediate protein delivery—which pathways are penetrable, Biochimica et Biophysica Acta 1798:2240-2248, incorporated by reference. Other embodiments are within the scope of the disclosure including, for example, activity sensors delivered as pro-analytes bearing a blocking group or other molecular cap that must be cleaved off (e.g., enzymatically or chemically) within the body to activate the activity sensor.

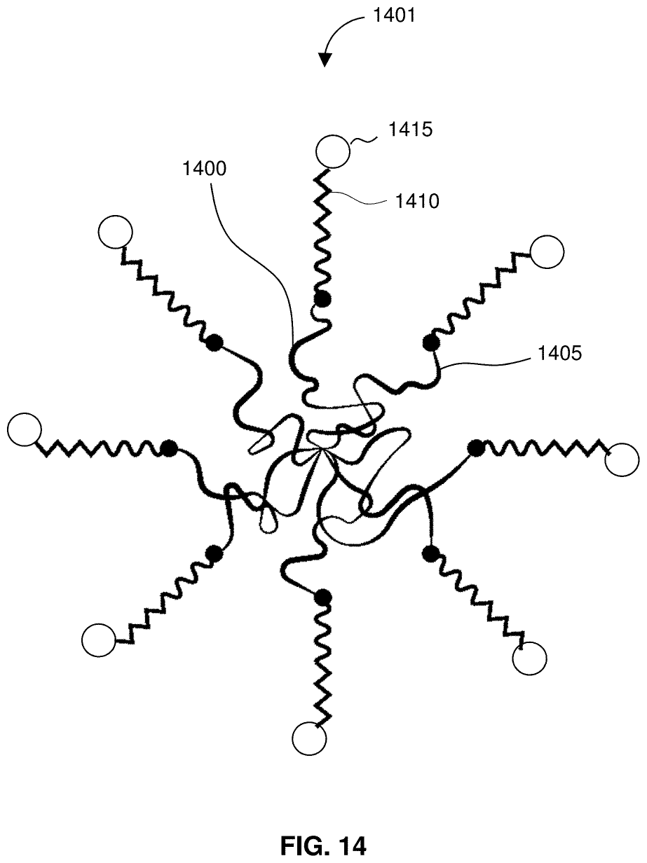

FIG. 14 shows a composition 1401 useful in methods of the disclosure. The composition 1401 preferably includes a plurality of activity sensors 1400 in which each activity sensor 1400 is linked to one or more blocking groups 1415. In the depicted embodiment, the blocking groups 1415 are linked to detectable reporters 1410. The detectable reporters 1410 may further be linked to a carrier 1405 such as a polymer scaffold.

In some embodiments, the activity sensor is bound to a molecular cap or blocking group. The polypeptide chains are protease substrates that would release the detectable reporter upon encountering the proteases, but for the blocking group sterically hindering access of the chains to the proteases. The composition is delivered into the body and the active form of the activity sensor is not presented at the target site or compartment until the molecular cap or blocking group is liberated. This embodiment can make use of enzymatic activity within the body to release the cap or blocking group, or a chemical environment of the body can liberate the cap or blocking group (e.g., the cap is released by acid-catalyzed hydrolysis only upon passage through the digestive system). Alternatively, an exogenous enzyme that liberates the cap or blocking group can be delivered such that the active form of the activity sensor is not present until both the composition and the exogenous enzyme have been delivered into the patient and interacted with each other. Other embodiments are within the scope of the disclosure.

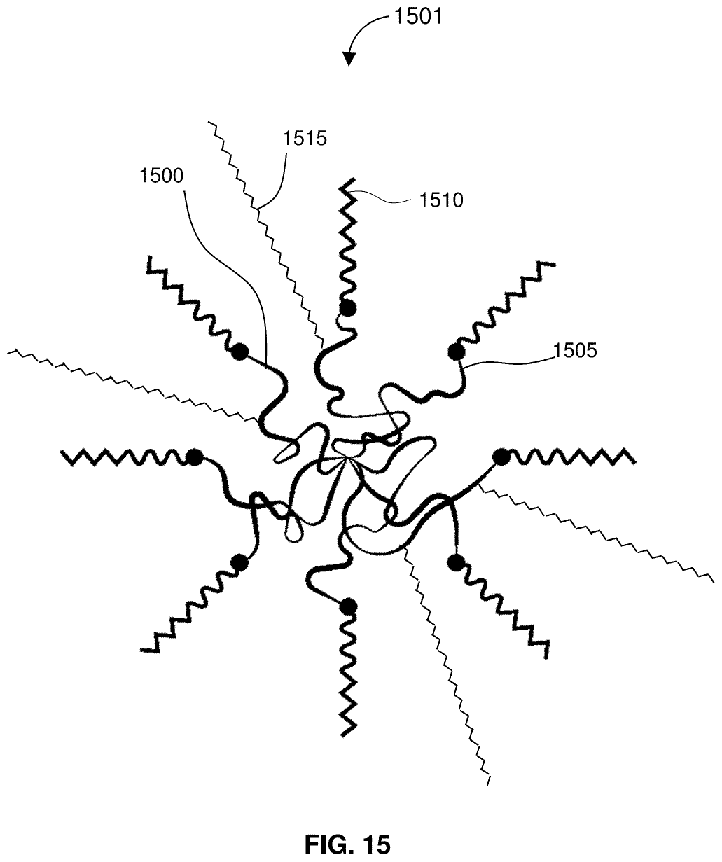

FIG. 15 shows a lipid-modified composition 1501 useful in methods of the disclosure. The composition 1501 preferably includes a plurality of activity sensors 1500 in which each activity sensor 1500 is linked to one or more lipid 1515. In the depicted embodiment, the lipids 1515 are linked to detectable reporters 1510. The detectable reporters 1510 may further be linked to a carrier 1505 such as a polymer scaffold. The use of lipids may protect the activity sensor from sequestration and decay in a predominantly aqueous environment, may promote accumulation of the activity sensors in adipose tissue, or may aid the activity sensors 1500 in evading immune detection and clearance. In some embodiments, the composition 1501 is delivered into adipose tissue. The detectable reporters 1510 are liable to cleavage in the presence of disease-associate enzymes that have been established to have elevated expression in association with known disease comorbidities known to be comorbid with obesity. The composition 1501 may be useful in evaluating overall health for an obese patient by detecting (or not detecting and thus ruling out) other disease conditions such as diabetes, liver disease, heart disease, etc.

Compositions and methods of the disclosure preferably use one or any combination of the pro-analytes shown or disclosed herein and the disclosed modifications or side-chains on activity sensors may include in any combination. Compositions and methods of the disclosure may be used to detect and distinctly report different activities that are associated with a physiological state of interest as well as one or more additional comorbidities. For example, it is understood that liver disease is commonly comorbid with obesity. Compositions of the disclosure can distinctly report activities specific to both obesity and liver disease. Methods of the disclosure are useful to provide information about comorbid diseases such as obesity, diabetes, liver disease, heart disease, others, and combinations thereof. Measurements may be made from different parts of the body. Integration or comparison of data from different parts of the body may be used to give specificity. For example, multiple compositions of the disclosure may be administered at once or in series. The compositions may go to different tissues, organs, or bodily compartments (e.g., one goes to the liver, the other goes to fat tissue). Different signals are obtained from the multiple compositions, and the differences between the signals reveals relevant differences among the activities occurring within the body.

To illustrate comorbidities, compositions and methods herein may be used to detect a signal of activity from different parts body. Compositions and methods may be used to detect activity, for example, in the liver and in adipose tissue. Differences in the signals may reveals something about a physiological state of interest, or may reveal effects of drugs that exhibit different effects on different people. Thus the disclosure provides for understanding the site and type of a response. Compositions and methods herein give information about off-target effects and give a signal that may be orthogonal to other signals such as organ image. Some NASH drugs treat one of fibrotic or lipoapototic (NASH) effects. Such a drug may be anti-fibrotic or anti-NASH. A composition of the disclosure may be used to show which mechanism of action is dominant in a given patient, to aid in selecting an appropriate therapeutic. If a patient is affected by one dominant mechanism of action, the composition of the disclosure reveals that, allowing suitable treatment to be administered.

In another example, it may be found that when a patient with a tumor is treated with a checkpoint blockade, it is common for the tumor to increase in size thereafter. However, the size increase may be directly attributable to an effectiveness of, or ineffectiveness of, the checkpoint blockade. Where the checkpoint blockade is effective, an influx of immune cells may cause an increase of size of the tumor even while the tumor cells are being destroyed. Where the checkpoint blockade is not effective as against that patient, the increase in tumor size may be caused simply by the continued proliferation of the tumor cells. Compositions and methods of the disclosure provide a rapid and minimally-invasive test of activity that discriminates between those possible outcomes. Thus compositions and methods of the disclosure provide for the creation of a signal (which may include the release of a detectable reporter). The signal indicates the rate and location of specific activities within the body and may be complementary to or orthogonal to imaging.

Methods of the disclosure further provide a framework for determining activity sensors to include in a composition, i.e., to determine what set of proteases of interest should be addressable or addressed using a composition of the disclosure and thus selecting the relevant polypeptides 207 to include for the detectable reporters. For each protease that the composition is intended to interrogate, one specific activity sensor 200 may be included, that activity sensor preferably linked to a plurality (e.g., 8) of polypeptides 207 that include a cleavage site 221, e.g., a scissile bond susceptible to cleavage by that protease. The activity sensors 200 optionally include a pro-analyte moiety 215 that is removed by the process in the body. Using such activity sensors 200, methods 101 include detecting 121 the detectable reporters released from the activity sensors at the target site to thereby sense activity of the body at the target site.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. An analysis method, the method comprising the steps of:
administering a pro-analyte to a patient, wherein in situ processing in the body results in enrichment of detectable reporters from the pro-analyte in proportion to a health condition of the patient;
measuring the detectable reporters in a sample from the patient; and
correlating the measured reporters to the health condition in the patient.

2. The method of claim 1, wherein the pro-analyte is processed through multiple organ systems, resulting in enrichment of the detectable reporters, and wherein the method further comprises providing a report comprising health information concerning the patient.

3. The method of claim 1, wherein the sample is selected from an exhalant, urine, blood, saliva, stool, tissue, sputum or noninvasive imaging method.

4. The method of claim 1, wherein measuring the detectable reporters comprises imaging the sample, and analyzing with a computer an image obtaining in the imaging step.

5. The method of claim 4, wherein the measuring step further comprises distributing the sample on a strip of an ELISA or lateral-flow assay, using a mobile device to image the strip, and using an app on the mobile device to perform the analyzing step.

6. The method of claim 1, wherein said enrichment comprises release of said detectable reporters in the presence of a specific disease state.

7. The method of claim 6, wherein said disease state is organ- or tissue-specific.

8-11. (canceled)

12. The method of claim 1, wherein the measuring step comprises quantifying released reporters.

13. The method of claim 1, wherein the administering step comprises administering a first composition comprising a first set of pro-analytes and administering a second composition comprising a second set of pro-analytes to the patient.

14. The method of claim 13, wherein release of detectable reporters from the first set of pro-analytes is indicative of activity at a first organ, tissue, or bodily compartment, and release of detectable reporters from the second set of pro-analytes is indicative of activity at a second, organ, tissue, or bodily compartment.

15. The method of claim 1, further comprising performing non-invasive analysis of the patient.

16. The method of claim 1, wherein the pro-analyte is a carrier comprising a linker connected to one or more of said detectable reporters.

17. The method of claim 16, wherein said carrier is a multi-arm polyethylene glycol scaffold, a tissue- or organ-targeted antibody, or an alternate synthetic carrier.

18. The method of claim 1, wherein said detectable reporters are released via a condition-specific chemical reaction or chemical environment in the patient.

19. The method of claim 18, wherein said condition-specific chemical reaction is one that occurs in excess in the presence of disease but that is substantially absent in a healthy organ.

20. The method of claim 19, wherein said chemical reaction is an enzymatic reaction catalyzed by an enzyme that is present in a disease state but substantially absent in a healthy condition.

21. The method of claim 18, wherein said chemical reaction or environment is one that occurs primarily in an extracellular matrix or membrane of a diseased cell.

22. The method of claim 1, wherein the pro-analyte is administered as: a plurality of a first particle, each first particle comprising a plurality of a first detectable reporter releasably attached to a carrier; a plurality of a second particle, each second particle comprising a plurality of a second detectable reporter releasably attached to a carrier; and a plurality of a third particle, each third particle comprising a plurality of a third detectable reporter releasably attached to a carrier.

23. The method of claim 22, wherein the reporters are attached to the particles by a respective cleavage site of an enzyme, wherein at least one cleavage site is cleaved by an enzyme that exhibits greater activity under a disease state than under a non-disease state.

24. The method of claim 13, further comprising the step of measuring a rate of change in the amount of the detectable reporter between said first and second administering steps.
